(19) United States
(12) Patent Application Publication
(10) Pub. No.: US 2023/0346999 A1
(43) Pub. Date: Nov. 2, 2023

(54) DECONTAMINATION METHOD

(71) Applicant: MIXSCIENCE, BRUZ (FR)

(72) Inventors: Victor PAUMELLE, BRUZ CEDEX (FR); Typhaine MORISSET, BRUZ CEDEX (FR); Florian BLANCHET, BRUZ CEDEX (FR)

(73) Assignee: MIXSCIENCE, BRUZ (FR)

(21) Appl. No.: 17/925,730

(22) PCT Filed: May 20, 2021
(86) PCT No.: PCT/EP2021/063492

§ 371 Date: Nov. 16, 2022

(30) Foreign Application Priority Data
May 20, 2020 (FR) FR2005280

(51) Int. Cl. A61L 2/16; A23K 10/18; A23L 3/3571

(52) U.S. Cl. CPC A61L 2/16; A23K 10/18; A23L 3/3571

(57) ABSTRACT

A food composition including at least one strain of bacteria of the genus Bacillus, the strain being dispersed in the food composition, and a method for decontaminating or preventing surface contamination of a device for receiving food in which the food composition is brought into contact with the surface of the device.

The invention relates to a decontamination method, in particular for decontaminating food production lines.

The prevention, limitation or even eradication of cross-contamination of foodstuffs during their preparation or even their storage is a challenge for the food industry. In particular, Salmonella spp and Escherichia coli are the most dangerous bacteria for both humans and animals when they are found in food, either because they cause serious pathologies or because asymptomatic carriage can be a source of epidemics of dietary origin in humans.

Several routes of contamination of foodstuffs are known, such as the lack of heat treatment of foodstuffs, inappropriate storage conditions, contamination during transport, contamination by rodents, birds, or insects, the development of condensation, the presence of contaminated dust, staff hygiene, but also contamination via the multiplication of pathogens in all hot and humid environments or via the dissemination and persistent presence of these pathogens in food storage facilities.

There is therefore a need to decontaminate premises, machines, equipment and even the foodstuffs themselves.

Various techniques are already known from the state of the art that make it possible to decontaminate premises and food production lines, using organic acids or chemical compounds. However, such compounds are no longer desired by the consumer, who is looking for natural products that are safe for health. In addition, some of these compounds accelerate the wear of industrial tools.

Thus, the food industry has turned to other means of decontamination.

In order to limit contamination, it has been proposed to provide animals with probiotic compositions ingested via food and limiting, within the animal itself, the development of pathogenic bacteria potentially present in food, and thus limiting or even eradicating disease risks. This is particularly the case of application WO2009/155711, which describes the use of compounds secreted by lactic acid bacteria, with a view to preventing infections by salmonella or Escherichia coli. However, such methods only deal with the consequences of the contaminations and do not attack the origins of the contamination.

However, such compositions do not appear to be effective in providing hygiene and contamination pressure at a level below any risk to animal and human health.

In addition, such compositions do not meet the need for prevention or decontamination of finished products, tools and production lines, storage areas or even conveyance routes for food products.

Thus, the purpose of the invention is to overcome the drawbacks of the prior art.

One of the aims of the invention is to propose an effective means of limiting the contamination pressure, in particular by salmonella or E. coli, not only in food, but also in the buildings and materials where and with which such food is produced, stored and transported.

The invention therefore relates to the use of a food composition comprising at least one strain of bacteria of the genus Bacillus, in particular of the species Bacillus subtilis, said strain being dispersed in said food composition, for decontaminating or preventing contamination of surfaces, in particular the surfaces of devices for preparing, storing and conveying food, that is to say, devices intended to receive food, in particular animal feed.

The invention is based on the surprising observation made by the inventors that the use of a food comprising a strain of bacteria of the genus Bacillus, especially of the species Bacillus subtilis, not only makes it possible to prevent the development of pathogenic or undesirable bacteria within said food, but that this food itself can be used for contamination prevention, or as a product for decontaminating the surfaces with which it is in contact.

The composition according to the invention is more advantageous than the liquid solutions for the application of bacteria proposed in the prior art because it allows decontamination of all the surfaces of devices intended to receive food, which the liquid applications of decontamination solutions, such as atomization, spraying, misting, nebulization or thermal nebulization or even vaporization, do not allow within production lines. It is of course inconceivable to replace the food in the production line with a flow of liquid without damaging said animal feed production line. Thus, the only way to decontaminate such production lines is to use hatches or openings located in certain specific places.

However, due to certain hermetic sections that are impossible for the user to access, the applications of liquids by spraying, atomization or vaporization do not allow the spread of bacteria throughout the entire transit circuit of the food product. Only the food composition would be able to run through the entire production network. It is therefore advantageous to integrate bacteria therein exerting a decontaminating power.

In addition, food production is not stopped during decontamination, since the food product exerts a decontaminating power during its manufacture, whereas decontamination with a liquid solution requires the production line to be stopped and possibly dried before being brought back online.

Thus, economically, the use of the composition according to the invention is advantageous in terms of production and yield, since the surfaces of the devices are decontaminated during manufacture and not when stopped.

Another advantage of the composition according to the invention with respect to liquid applications is to provide, at the same time as the positive bacteria, nutrients (derived from the food) allowing the development of the latter. Consequently, the positive bacteria will be able to develop and exert a greater decontamination power on pathogenic bacteria.

In the invention, the terms “food” or “food composition” will be used uniformly to designate the same product.

In the invention, when reference is made to a pathogenic bacterium, reference is also made to an unwanted bacterium.

In the invention, a food or a food composition is used as a prevention or decontamination agent. This food is composed of one or more substances likely to be ingested and digested, and to be used for the nutrition of a living being. Thus, the food that is defined in the invention can take all possible solid or semi-solid forms, such as powders, including flours, granules, crumbs, pebbles, boluses, tablets, pastes, gels considered by those skilled in the art of the field in question to be solids, rehydrated foods, etc. However, the form of the food according to the invention is in no way a liquid form or a liquid intended for drinking.

The food according to the invention therefore consists of one or more substances that provide nutrient intake for humans or animals. They may therefore be substances consisting or essentially consisting of carbohydrates, lipids, proteins, or minerals, or a mixture of these two by two, or a mixture of these three by three, or a mixture of all of these. The food can be a simple mixture of simple elements (lipids, carbohydrates, proteins, minerals), one or more raw materials, i.e. initial products from agriculture (such as fruits, leaves, seeds, stems, bark or roots, etc.) or from mining extraction (such as clays, carbonates, etc.), in the raw, semi-processed or processed state, or a more complex product such as products derived from animals (flesh, soft organs, bones, cartilage, etc.), products derived from plants (fibers, seeds, flowers, roots, etc.) or even co-products derived from the food industry (cheese whey, spent grains from breweries, etc.), all of which may possibly be supplemented with food additives and/or processing aids. Thus, all forms of products consumable by humans or animals that are commercially available correspond to the food described in the invention. It should be noted that the foods mentioned above may, although consumable, not be consumed after use according to the invention, for various reasons (e.g.: rinsing batches).

The food used in the context of the invention comprises at least one strain of bacteria of the genus Bacillus, especially of the species Bacillus subtilis. This means that one or more bacteria of said strain are found in said food, in particular in a dispersed form, more or less homogeneously. It is possible to disperse bacteria of a strain within the food by performing simple mixing so that the bacteria of the strain are distributed evenly from the surface of the food to its core. To obtain this dispersion, a step of heating, humidification, grinding, or any other type of pretreatment, that is to say, treatment prior to implementing the invention, alone or in combination, may be deemed necessary by those skilled in the art.

Advantageously, this will be referred to as a homogeneous dispersion so that macroscopically, the bacteria of the Bacillus strain are present all throughout the material of the food from its surface to its core, so that on average a bacterium or a group of bacteria is equidistant from another bacterium or another group of bacteria and so that it is guaranteed that a minimum concentration of bacteria can be measured by random sampling in the food.

In the invention, “strain of bacteria” means all the individuals (bacteria) resulting from the multiplication of a bacterial colony that in turn results from an isolation by subculture, this set being essentially homogeneous, that is to say, the very great majority of bacteria that make up the strain have the same genotype. In other words, a strain is a part of a bacterial species that differs from other bacteria of the same species by a minor but identifiable difference. A strain is also defined as a population of bacteria that descended from a single organism or pure isolate culture. Strains of the same species may differ slightly from each other in many ways.

In the invention, “at least one strain of bacteria of the genus Bacillus” means that the food may contain a single strain of bacteria according to the definition above, or two, or three or more strains of bacteria, all these strains, although the species may differ, belonging to the genus Bacillus.

It is important to note that it is not necessary to take special precautions during the manufacture of the food according to the invention, in particular bacteriological precautions. Indeed, since the food contains bacteria of the genus Bacillus, in particular of the species Bacillus subtilis, said food does not require the nutritional substances that constitute the food themselves to be decontaminated. In any event, it is, however, essential that the food does not contain any anti-bacterial agent (bacteriostatic or bacteriolytic) so as not to kill bacteria of the genus Bacillus contained in that food.

The food described above can therefore, within the scope of the invention, be used to decontaminate or prevent the contamination of surfaces. This means that the aforementioned food is capable, when it is applied to said contaminated surface, of eliminating bacterial contamination, and in particular of eliminating bacterial contamination capable of causing disorders in humans or animals, such as contamination by salmonella or E. coli. This decontamination can be total, so that once the food according to the invention is brought into contact with the contaminated surface, the result is that bacteria of the salmonella or E. coli type eventually disappear. The decontamination can also be partial, that is to say, bringing the food according to the invention into contact with a contaminated surface will not eliminate all of the salmonella or E. coli present, but will maintain or reduce their numbers so that the amount of salmonella or E. coli will be such that the risks to human or animal health will be limited.

The food can also prevent contamination, which means that if a surface that is not contaminated, for example with salmonella or E. coli, is initially treated, or brought into contact with the food according to the invention, when the surface later encounters salmonella or E. coli, the latter will not be able to develop in such a way as to become so numerous that they would represent a risk for animal or human health or, in the event of proven development of these pathogenic or undesirable bacteria, this development will be less significant than without the food according to the invention.

Thus, the aforementioned decontamination can be likened to a “cleaning” of a contaminated surface, while the contamination prevention can be likened to a “protection” against contamination, or against the expansion of residual or minimal contamination that is already present.

In the invention, reference is made to contamination prevention or surface decontamination. Here, “surface” means all the internal or external surfaces located in a building where a food product is manufactured, in particular a production line, or in a building where it is used, in particular a feeding screw in animal husbandry. This therefore includes, but is not limited to, the walls, windows, doors, ceilings of the building, including the various moldings, complaints, beams, hatches, and all other constituent elements of the building that may be visible. This also includes, but is not limited to, all internal or external surfaces directly in contact with the food, devices such as, for example, tubes, silos, storage or guide cones, presses, crushers, screw or belt conveyors, pneumatic transfer installations, vertical or horizontal coolers, mixers and their dosing installations such as bag dumpers, kneaders, reception pits and their filters, bulk loading systems and their unloading hatches, sandblasters, sifters, crumbling devices (crumblers), lump breaking devices (lump breakers), automatic samplers, coating devices (coaters), sprayers, extruders, ovens, cookers, extractors, refrigerators, drying ovens, etc., all of these devices being considered individually and with their constituent parts.

Advantageously, the invention relates to the use of the aforementioned food composition, said composition further comprising at least one strain of lactic acid bacteria.

The inventors have noticed that the food that is used for the decontamination or the contamination prevention as defined in the invention is all the more effective if one combines at least one strain of bacteria of the genus Bacillus, in particular of the species Bacillus subtilis, and at least one strain of lactic acid bacteria.

Thus, the invention advantageously relates to the use of a food composition comprising at least one strain of bacteria of the genus Bacillus, in particular of the species Bacillus subtilis, and at least one strain of lactic acid bacteria, said strains being dispersed in said food composition, for decontaminating or preventing contamination of surfaces, in particular the surfaces of devices for preparing, storing and conveying food, in particular animal feed.

Advantageously, the invention relates to the aforementioned use, where said strain of bacteria is present at a rate of at least 10⁴ cfu per gram of food composition.

In the invention, “cfu” or “colony-forming unit” means a unit used to estimate the number of viable bacteria in a sample. Viability is defined as the ability to multiply, i.e. to divide. Counting with colony-forming units requires culturing the microorganisms and thus counts only viable cells.

In this advantageous embodiment, the composition according to the invention is in dehydrated or non-aqueous form (in solid or semi-solid form), and said composition preferably comprises from 10⁴ to 10¹¹ viable bacterial colonies of Bacillus per g of composition, or cfu per g of composition, for each of the strains when the composition comprises at least two strains.

In the invention, “from 10⁴ to 10¹¹ viable bacteria, or cfus” means: approximately 10⁴, approximately 5.10⁴, approximately 10⁵, approximately 5.10⁵, approximately 10⁶, approximately 5.10⁶, approximately 10⁷, approximately 5.10⁷, approximately 10⁸, approximately 5.10⁸, approximately 10⁹, approximately 5.10⁹, approximately 10¹⁰, approximately 5.10¹⁰ or approximately 10¹¹ viable bacteria.

Viable bacteria are expressed in grams of bacterial culture. A person skilled in the art easily knows how to determine this number of bacteria, in particular by counting either manually (using a Malassez blade), or by using an automatic cell counter, or by dilution, then inoculation on agar and counting, in particular visual, of the colonies, or by measuring the optical density.

In an advantageous embodiment where the food composition comprises both bacteria of the genus Bacillus, in particular of the species Bacillus subtilis, and lactic acid bacteria, each of these bacteria is present in a proportion of at least 10⁴ cfu per gram of food composition, in particular in a proportion of 10⁴ to 10¹¹ cfu per g of food composition.

Advantageously, said one or more strains of bacteria of the genus Bacillus, in particular Bacillus subtilis, are in vegetative or spore form, or both.

Advantageously, the invention relates to the use as defined above, where said strain of bacteria of the genus Bacillus is chosen from one of the following strains:
strain NOL01, deposited with the Collection Nationale de Cultures de Microorganismes [National Collection of Microorganism Cultures] (CNCM, 25-28 rue du Docteur Roux 75724 Paris Cedex 15, France) on Mar. 14, 2012 under number CNCM 1-4606,
strain NOL02, deposited with the CNCM on Jan. 21, 2016 under number CNCM 1-5043, and
strain NOL03, deposited with the CNCM on Mar. 14, 2012 under number CNCM 1-4607,

or any of the strains of bacteria of the genus Bacillus belonging to the same operational taxonomic unit (OTU) as said NOL01 strain, or said NOL02 strain or said NOL03 strain,

or a mixture of two or more of these strains, that is to say, NOL strains described above or strains belonging to said OTUs.

In the invention, “a Bacillus strain belonging to the same operational taxonomic unit (OTU) as said NOL01 strain, or said NOL02 strain or said NOL03 strain” or “a similar strain” to NOL01, or NOL02, or NOL03, or “strains having a very similar” or “very close” genome to NOL01, or NOL02, or NOL03, means all strains of Bacillus, in particular Bacillus subtilis, having at least 97% of genomic sequences similar to the sequence of genes encoding the 16S rRNA of the NOL01, or NOL02, or NOL03 strain.

In biology, the operational taxonomic unit (OTU) is the basic unit of phylogenetic analysis used to group phylogenetically close individuals. The DNA sequences of a given gene, which then serves as a taxonomic marker, are grouped together by data partitioning (clustering) according to their identity. One gene that is primarily used (reference gene) is 16S rRNA for bacteria. Thus, the OTUs are defined on the basis of an identity threshold chosen by those skilled in the art, which is commonly 97%.

Advantageously, the invention relates to the use as defined above, where said strain of lactic acid bacteria is chosen from one of the following strains:
strain NOL11, deposited with the CNCM on Mar. 14, 2012 under number CNCM 1-4609,
one of the strains of lactic acid bacteria belonging to the same operational taxonomic unit as said NOL11 strain,
and a mixture of two or more of these strains, that is to say, the NOL11 strain or strains belonging to said OTU.

Thus, in an advantageous embodiment, the invention relates to the aforementioned use, where said food composition comprises:
at least one strain of bacteria of the genus Bacillus, chosen from strains NOL01, NOL02, or NOL03, or one of the strains of bacteria of the genus Bacillus belonging to the same OTU as said NOL01 strain, or said NOL02 strain or said NOL03 strain, and
at least one strain of lactic acid bacteria, chosen from the NOL011 strain, or any of the strains of lactic acid bacteria belonging to the same OTU as said NOL11 strain,
and a mixture of two or more of these strains, that is to say, NOL strains or strains belonging to said OTUs.

Even more advantageously, the invention relates to the above-mentioned use, where said composition comprises at least one of the following combinations of bacteria:
NOL01 and NOL11,
NOL02 and NOL11,
NOL03 and NOL11,
NOL01, NOL02 and NOL11,
NOL01, NOL03 and NOL11,
NOL02, NOL03 and NOL11,
NOL01, NOL02, NOL03 and NOL11.

Advantageously, the invention further relates to the aforementioned use where said composition comprises at least one of the following combinations of bacteria:

TABLE 1

| | | | | | OTU | OTU | OTU | OTU |
|---|---|---|---|---|---|---|---|---|
| | NOL01 | NOL02 | NOL03 | NOL11 | NOL01 | NOL02 | NOL03 | NOL11 |
| 1 | X | | | X | | | | |
| 2 | X | | | | | | | X |
| 3 | | | | X | X | | | |
| 4 | | | | | X | | | X |
| 5 | | X | | X | | | | |
| 6 | | X | | | | | | X |
| 7 | | | | X | | X | | |
| 8 | | | | | | X | | X |
| 9 | | | X | X | | | | |
| 10 | | | X | | | | | X |
| 11 | | | | X | | | X | |
| 12 | | | | | | | X | X |
| 13 | X | X | | X | | | | |
| 14 | X | X | | | | | | X |
| 15 | X | | | X | | X | | |
| 16 | X | | | | | X | | X |
| 17 | | X | | X | X | | | |
| 18 | | X | | | X | | | X |
| 19 | | | | X | X | X | | |
| 20 | | | | | X | X | | X |
| 21 | X | | X | X | | | | |
| 22 | X | | X | | | | | X |
| 23 | X | | | X | | | X | |
| 24 | X | | | | | | X | X |
| 25 | | | X | X | X | | | |
| 26 | | | X | | X | | | X |
| 27 | | | | X | X | | X | |
| 28 | | | | | X | | X | X |
| 29 | | X | X | X | | | | |
| 30 | | X | X | | | | | X |
| 31 | | X | | X | | | X | |
| 32 | | X | | | | | X | X |
| 33 | | | X | X | | X | | |
| 34 | | | X | | | X | | X |
| 35 | | | | X | | X | X | |
| 36 | | | | | | X | X | X |
| 37 | X | X | X | X | | | | |
| 38 | X | X | X | | | | | X |
| 39 | | X | X | X | X | | | |
| 40 | | X | X | | X | | | X |
| 41 | X | | X | X | | X | | |
| 42 | X | | X | | | X | | X |
| 43 | X | X | | X | | | X | |
| 44 | X | X | | | | | X | X |
| 45 | X | | | X | | X | X | |
| 46 | X | | | | | X | X | X |
| 47 | | X | | X | X | | X | |
| 48 | | X | | | X | | X | X |
| 49 | | | X | X | X | X | | |
| 50 | | | X | | X | X | X | |
| 51 | | | | X | X | X | X | |
| 52 | | | | | X | X | X | X |

Advantageously, the invention relates to the aforementioned use, where said surfaces are contaminated or contaminable by enterobacteriaceae, in particular by strains of the genus Salmonella or Escherichia, in particular bacteria of the species Salmonella enterica or Escherichia coli.

The abovementioned food composition is particularly useful for decontaminating, or preventing contamination, or even controlling contamination of surfaces that are contaminated or that could be contaminated by enterobacteriaceae.

The Enterobacteriaceae family is defined by the following characteristics:
Gram-negative bacilli (2 to 4 microns long by 0.4 to 0.6 microns wide),
mobile bacteria with peritrichous cilia or immobile bacteria,
growing on ordinary culture media,
essentially aerobic bacteria, or optionally anaerobic,
glucose-fermenting bacteria with or without gas production,
bacteria reducing nitrates to nitrites,
oxidase-negative bacteria.

The Enterobacteriaceae family is a very heterogeneous family in terms of pathogenesis and ecology. The species that make up this family are in fact either parasitic (Shigella, Yersinia pestis), or commensals (Escherichia coli, Proteus mirabilis, Klebsiella sp), or even saprophytes (Serratia sp, Enterobacter sp).

The Salmonella spp. are enterobacteriaceae whose essential characteristics are not to ferment lactose and not to produce urease. The Salmonella spp. are parasites of humans, mammals (rodents), birds (poultry) and cold-blooded animals (reptiles). They are responsible, after oral penetration, for numerous infections (salmonellosis), in particular typhoid and paratyphoid fevers (notifiable diseases no. 1), gastroenteritis and collective food poisoning (notifiable diseases no. 2).

The main mode of contamination in humans is ingestion from water (Salmonella enterica serotype Typhi in particular) or foods (e.g. dairy products, eggs, meat).

Non-typhoid Salmonella spp. (Salmonella enterica serotype Typhimurium, serotype Enteritidis, serotype Dublin, etc.), ubiquitous, is ingested with a contaminated drink or food (sporadic cases) or after fecal-oral contamination, often by dirty hands (community epidemics). This can result in purely digestive infections, gastroenteritis. These result in diarrhea, vomiting and fever. Their progression is generally benign. Some subjects remain healthy carriers of Salmonella spp. in their digestive tract and in certain circumstances (food profession, for example) can disseminate their strain.

Some strains of E. coli are associated with diarrhea and are clearly enteropathogenic (EPEC) owing to particular adhesion properties. They are neither enterotoxin-secreting nor enteroinvasive. They form pili, which form “bundles” that attach to the villi of enterocytes. The villi are gradually destroyed (“attachment-erasing”). The cytoskeleton of enterocytes is altered and a water leak occurs very quickly, the biochemical mechanism of which has not been fully elucidated.

In another aspect, the invention relates to a method for decontaminating or preventing contamination of surfaces of a device for receiving food, said method comprising a step of bringing a food composition into contact with the surfaces of said device, said food composition comprising at least one strain of bacteria of the genus Bacillus, in particular of the species Bacillus subtilis, said strain being dispersed in said food composition.

In the aforementioned method or use, “device for receiving food” means any device for preparing, conveying or storing food, including the walls, floor, windows, and ceiling of a room or building containing one or more devices for preparing, transporting or storing food.

The objective is therefore to bring a device for receiving food into contact in order to decontaminate it or to protect it from potential contamination, or even to limit the development of an existing contamination. A food composition can be brought into contact with the surfaces to be decontaminated, or for which contamination prevention is desired, using tools facilitating the application, in particular using tools of the sandblasting type.

The invention advantageously relates to the aforementioned method, where said food composition further comprises at least one strain of lactic acid bacteria.

Thus, the invention relates to a method for decontaminating or preventing contamination of surfaces of a device for receiving food, said method comprising a step of bringing a food composition into contact with the surfaces of said device, said food composition comprising at least one strain of bacteria of the genus Bacillus, in particular of the species Bacillus subtilis, and a strain of lactic acid bacteria, said strains being dispersed in said food composition.

In an advantageous embodiment, the invention relates to the method mentioned above, where said strain of bacteria is present at a rate of at least 10⁴ cfu per gram of food composition.

In this advantageous embodiment, the composition according to the invention is in dehydrated or non-aqueous form (in solid or semi-solid form), and said composition preferably comprises from 10⁴ to 10¹¹ viable bacterial colonies of Bacillus per g of composition, or cfu per g of composition, for each of the strains when the composition comprises at least two strains.

In the invention, “from 10⁴ to 10¹¹ viable bacteria, or cfu” means: approximately 10⁴, approximately 5.10⁴, approximately 10⁵, approximately 5.10⁵, approximately 10⁶, approximately 5.10⁶, approximately 10⁷, approximately 5.10⁷, approximately 10⁸, approximately 5.10⁸, approximately 10⁹, approximately 5.10⁹, approximately 10¹⁰, approximately 5.10¹⁰ or approximately 10¹¹ viable bacteria.

In an advantageous embodiment where the food composition comprises both bacteria of the genus Bacillus, in particular of the species Bacillus subtilis, and lactic acid bacteria, each of these bacteria is present in a proportion of at least 10⁴ cfu per gram of food composition, in particular in a proportion of 10⁴ to 10¹¹ cfu per g of food composition.

Advantageously, said one or more strains of bacteria of the genus Bacillus, in particular Bacillus subtilis, are in vegetative or spore form, or both.

Advantageously, the invention relates to the method mentioned above, where said strain of bacteria of the genus Bacillus is chosen from one of the following strains:
strain NOL01, deposited with the Collection Nationale des Cultures de Microorganismes (CNCM, 25-28 rue du Docteur Roux 75724 Paris Cedex 15, France) on Mar. 14, 2012 under number CNCM 1-4606,
strain NOL02, deposited with the CNCM on Jan. 21, 2016 under number CNCM 1-5043, and
strain NOL03, deposited with the CNCM on Mar. 14, 2012 under number CNCM 1-4607,

or any of the strains of bacteria of the genus Bacillus belonging to the same operational taxonomic unit (OTU) as said NOL01 strain, or said NOL02 strain or said NOL03 strain,

or a mixture of two or more of these strains, that is to say, NOL strains or strains belonging to said OTUs.

Advantageously, the invention relates to the method mentioned above, where said strain of lactic acid bacteria is chosen from one of the following strains: the NOL11 strain, deposited with the CNCM on Mar. 14, 2012 under number CNCM 1-4609, and one of the strains of lactic acid bacteria belonging to the same operational taxonomic unit as said NOL11 strain,

or a mixture of two or more of these strains, that is to say, NOL strains or strains belonging to said OTUs.

The invention also relates to a food composition in which is dispersed at least one strain of bacteria of the genus Bacillus, notably Bacillus subtilis.

Advantageously, the invention relates to the aforementioned food composition, in which is dispersed at least one strain of bacteria of the genus Bacillus, notably Bacillus subtilis, and at least one strain of lactic acid bacteria.

Even more advantageously, the composition mentioned above is such that said strain of bacteria of the genus Bacillus is chosen from one of the following strains:
strain NOL01, deposited with the CNCM on Mar. 14, 2012 under number CNCM 1-4606,
strain NOL02, deposited with the CNCM on Jan. 21, 2016 under number CNCM 1-5043, and
strain NOL03, deposited with the CNCM on Mar. 14, 2012 under number CNCM 1-4607,

or any of the strains of bacteria of the genus Bacillus belonging to the same operational taxonomic unit (OTU) as said NOL01 strain, or said NOL02 strain or said NOL03 strain,

or a mixture of two or more of these strains.

Furthermore, advantageously, the composition is such that said strain of lactic acid bacteria is chosen from one of the following strains: the NOL11 strain, deposited with the CNCM on Mar. 14, 2012 under number CNCM 1-4609, and one of the strains of lactic acid bacteria belonging to the same operational taxonomic unit as said NOL11 strain, or a mixture of two or more of these strains.

Advantageously, the invention relates to the food composition mentioned above, where said strain of bacteria is present at a rate of at least 10⁴ cfu per gram of food composition, that is to say, where said at least one strain of Bacillus is present at a rate of at least 10⁴ cfu per gram of food composition, and where appropriate, said strain of lactic acid bacteria is present at a rate of at least 10⁴ cfu per gram of food composition.

Advantageously, said one or more strains of bacteria of the genus Bacillus, notably Bacillus subtilis, is in vegetative or spore form, or both.

Advantageously, the invention relates to the food composition as defined above, where said strain of bacteria of the genus Bacillus is chosen from one of the following strains:
the NOL01 strain, deposited with the Collection nationale des cultures de microorganismes (CNCM, 25-28 rue du Docteur Roux 75724 Paris Cedex 15, France) on Mar. 14, 2012 under number CNCM 1-4606,
strain NOL02, deposited with the CNCM on Jan. 21, 2016 under number CNCM 1-5043, and
strain NOL03, deposited with the CNCM on Mar. 14, 2012 under number CNCM 1-4607,

or any of the strains of bacteria of the genus Bacillus belonging to the same operational taxonomic unit (OTU) as said NOL01 strain, or said NOL02 strain or said NOL03 strain,

or a mixture of two or more of these strains, that is to say, NOL strains or strains belonging to said OTUs.

Advantageously, the invention relates to the food composition mentioned above, where said strain of lactic acid bacteria is chosen from one of the following strains:
strain NOL11, deposited with the CNCM on Mar. 14, 2012 under number CNCM 1-4609,
one of the strains of lactic acid bacteria belonging to the same operational taxonomic unit as said NOL11 strain,
and a mixture of two or more of these strains, that is to say, NOL strains or strains belonging to said OTUs.

The invention will be better understood in the light of the following examples and drawings.

### BRIEF DESCRIPTION OF THE FIGURES

FIG. 1 schematically shows a device after application of food compositions on contaminated boxes, with X the sampling zone and Y the sedimentation zone of the composition.

FIG. 2 is a graph showing the amount of Salmonella Typhimurium in Log₁₀ CFU/g as a function of time in hours for a pig feed-type composition, comprising (B) or not comprising (A) the Bacillus and lactic acid bacteria (Test 1).

FIG. 3 is a graph showing the amount of E. coli in Log₁₀ CFU/g as a function of time in hours for a pig feed-type composition, comprising (B) or not comprising (A) the Bacillus and lactic acid bacteria (Test 3).

FIG. 4 is a graph showing the amount of Salmonella Enteritidis in Log₁₀ CFU/g as a function of time in hours for a ground corn-type composition, comprising (B) or not comprising (A) the Bacillus and lactic acid bacteria (Test 5).

FIG. 5 is a graph showing the amount of Salmonella Typhimurium in Log₁₀ CFU/g as a function of time in hours for a soy meal-type composition, comprising (B) or not comprising (A) the Bacillus and lactic acid bacteria (Test 8).

FIG. 6 is a graph showing the evolution after 24 hours of the quantity of Salmonella Typhimurium in Log₁₀ CFU/g as a function of time after the application of a food composition, comprising (1.) or not comprising (2.) the bacteria according to the invention (NOF01-03 and NOF11), over 6 series of tests: A and B crushed corn matrix, C and D sow feed matrix, or E and F without feed matrix.

### EXAMPLES

### Example 1: In Vitro Tests

The objective of these experiments was to test the ability of the food composition according to the invention to prevent, reduce or control the expansion of enterobacteriaceae in vitro.

### A. Preparation of the Inocula

Before use, the peptone water necessary for the preparation and counting of the cultures as well as the tap water used were sterilized at 121° C. for 20 minutes to avoid the presence of unwanted microorganisms.

Frozen isolates of pathogens, S. enterica or E. coli, were thawed and then dissolved in a culture medium for brain-heart infusion with yeast extracts (3.7% medium for brain-heart infusion (Merck, Darmstadt, Germany); 0.1% yeast extracts (Merck)) at 37° C. for 24 hours under aerobic conditions and without stirring. The inocula were then diluted in peptone water (0.85% sodium chloride (VWR, Langenfeld, Germany); 0.1% peptone (G-Science, Saint Louis, USA)) to reach an initial concentration of 1×10⁶ CFU/mL.

The initial concentrations in S. enterica or E. coli were measured by counting according to the Pasteurian method on agar for brain-heart infusion with yeast extracts, after incubation for 24 hours at 37° C.

### A. Preparation of the Food Matrices

To prepare the samples comprising the NOL01, NOL02, NOL03 and NOL11 strains, the bacteria were mixed for 5 minutes (TopMix94323, Heidolph) in the food preparation so as to reach a minimum concentration of 1×10⁶ CFU/g in Bacillus subtilis (NOL01-NOL03) and minimum 1×10⁶ CFU/g in Lactococcus lactis (NOL11).

The different matrices were then incorporated or not in water corresponding to the recommendations of the feed manufacturers as to their use:

TABLE 2

| | Mass factor of water incorporation to obtain the |
|---|---|
| Food preparation | preparation, matrix base = 1 |
| Pig feed | 2.75 |
| Crushed corn | 0.00 |
| Soy meal | 0.00 |

The pig feed used in this example is a feed whose composition is given in the following table:

TABLE 3

| | Incorporation rate (by mass relative to |
|---|---|
| Components | the total mass of of the feed) |
| Wheat | 50.77% |
| Peas | 14.38% |
| Wheat bran | 12.92% |
| Corn | 9.85% |
| Sunflower meal | 5.13% |
| Salt | 2.00% |
| Wheat gluten | 2.00% |
| Calcium carbonate | 1.63% |
| Water | 0.50% |
| Lysine Sulfate 70% | 0.43% |
| Vitamin and mineral premix | 0.20% |
| Sodium sulphate | 0.10% |
| L-threonine | 0.07% |
| DL-methionine | 0.02% |

For each sample, 2.5 g of food was weighed (Precision Series, Fisher

Scientific), in a sterile 120 mL jar (Sarstedt, Nümbreccht, Germany) from the same prepared pool. A jar corresponding to a time step for measuring the pathogens in a sample. The samples were then incorporated into sterilized water to model degraded process, storage and use conditions, at the rate of 2 mL of sterilized water per jar.

250 μL of inoculum of S. enterica or E. coli was added to the treated samples and to the control samples using a pipette (Finnpipette F2, Thermoscientific). Each sample was vortexed for 1 minute to obtain a homogeneous mixture (TopMix94323, Heidolph). The samples were then incubated under aerobic conditions in an incubator (BE500, Memmert) for a period of up to 7 days at 25° C. to model the ambient temperature in animal husbandry, in a production plant, or in a storage and transport area.

For technical reasons related to the performance of part of the counts in the outside analysis laboratory, the quantities of each matrix were multiplied by 10 during the performance of some experiments. Therefore, for each sample, jars of 120 mL containing 25 g of food are used, to which we add 20 mL of sterilized water and 2.5 mL of inoculum of S. enterica or E. coli.

### A. Pathogen Tracking

The counting of pathogenic bacteria was carried out at time 0 (T0), then at different time steps depending on the experiments carried out. At each time, the samples were analyzed to count S. enterica or E. coli according to at least one of the following reference methods:
Standard ISO 21528-2:2017 adapted—Microbiology of the food chain—Horizontal method for the detection and enumeration of Enterobacteriaceae—Part 2: Colony-count technique
Standard ISO 6579-1:2017 Microbiology of the food chain—Horizontal method for the detection, enumeration and serotyping of Salmonella—Part 1: Detection of Salmonella spp.
Standard ISO 16649-2:2001 Microbiology of human and animal feeding stuffs—Horizontal method for enumeration of beta-glucuronidase-positive Escherichia coli—Part 2: Colony-count technique at 44° C. using 5-bromo-4-chloro-3-indolyl beta-D-glucuronide.

### A. Statistical Analysis of Results

In view of the small number of samples during each test, the effect of the treatment with the food according to the invention on the quantity of pathogen at each time step and for each test is measured by the Wilcoxon test with a significance threshold set at 5% (p value less than or equal to 0.05), and a trend threshold set at 10% (p-value less than or equal to 0.1).

### A. Summary of the Tests Carried Out

TABLE 4

| | | | | Samples |
|---|---|---|---|---|
| | | | Count | per |
| Test | Matrix | Pathogen | standard | treatment |
| 1 | Pig feed | Salmonella | ISO | 5 |
| | | Typhimurium | 6579-1: 2017 | |
| 2 | Pig feed | Salmonella | ISO | 3 |
| | | Typhimurium | 21528-2: 2017 | |
| 3 | Pig feed | Escherichia | ISO | 5 |
| | | coli | 16649-2: 2001 | |
| 4 | Pig feed | Escherichia | ISO | 3 |
| | | coli | 21528-2: 2017 | |
| 5 | Crushed corn | Salmonella | ISO | 3 |
| | | Enteritidis | 21528-2: 2017 | |
| 6 | Soy meal | Salmonella | ISO | 6 |
| | | Typhimurium | 21528-2: 2017 | |

### A. Summary of Test Results

TABLE 5

| | | | | Effect | |
|---|---|---|---|---|---|
| | | | | produced | |
| | | | | at the | Mean |
| | | | Final | final time | difference |
| | | | time | (test | Control − Test |
| Test | Matrix | Pathogen | (hours) | p-value) | (Log₁₀ CFU/g) |
| 1 | Pig feed | Salmonella | 120 | 0.010 | 3.9 |
| | | Typhimurium | | | |
| 2 | Pig feed | Salmonella | 120 | 0.100 | 2.1 |
| | | Typhimurium | | | |
| 3 | Pig feed | Escherichia | 48 | 0.060 | 3.1 |
| | | coli | | | |
| 4 | Pig feed | Escherichia | 120 | 0.100 | 1.3 |
| | | coli | | | |
| 5 | Crushed corn | Salmonella | 48 | 0.100 | 3.6 |
| | | Enteritidis | | | |
| 6 | Soy meal | Salmonella | 120 | 0.026 | 1.9 |
| | | Typhimurium | | | |

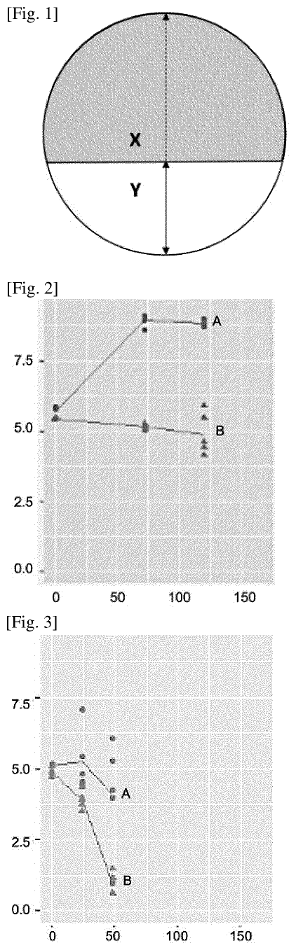

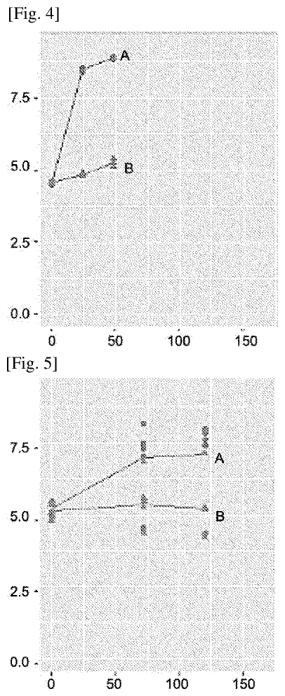

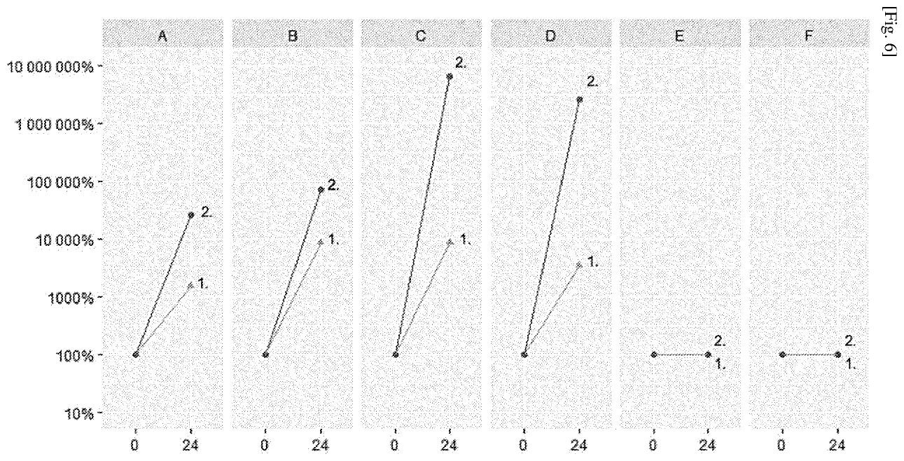

The results are presented in FIGS. 2 to 5.

These different results in vitro highlight a difference in the development of pathogens greater than 1 Log₁₀ CFU/g owing to the use of Bacillus and lactic acid bacteria. This effect is also present in matrices favorable to the development of enterobacteriaceae, which in practice are often the source of new contaminations in cascade.

The industrial use of Bacillus and lactic acid bacteria in food compositions is therefore promising. Indeed, the contact of these Bacillus and lactic acid bacteria with surfaces of devices for preparing, storing and conveying food via food compositions as defined in the invention will allow these Bacillus and lactic acid bacteria to spread on said surfaces in a preferred manner, thus limiting the proliferation of enterobacteria and therefore reducing the risk of subsequent recontamination.

### Example 2: Surface Test

Objective: demonstrate an effect of a food composition containing at least one Bacillus on controlling surface contamination. For practical reasons, the performance of this protocol can be adapted.

### A. Preparation of the Inocula

Before use, the peptone water necessary for the preparation and counting of the cultures as well as the tap water used are sterilized at 121° C. for 20 minutes to avoid the presence of unwanted microorganisms.

Frozen isolates of pathogens, S. enterica or E. coli, are thawed and then dissolved in a culture medium for brain-heart infusion with yeast extracts (3.7% medium for brain-heart infusion (Merck, Darmstadt, Germany); 0.1% yeast extracts (Merck)) at 37° C. for 24 hours under aerobic conditions and without stirring. The inocula are then diluted in peptone water (0.85% sodium chloride (VWR, Langenfeld, Germany); 0.1% peptone (G-Science, Saint Louis, USA)) to reach an initial concentration of 1×10² CFU/mL.

The initial concentrations in S. enterica or E. coli are checked by counting according to the Pasteurian method on agar for brain-heart infusion with yeast extracts, after incubation for 24 hours at 37° C.

### A. Preparation of the Food Matrices

To prepare the test samples, that is to say, the food compositions according to the invention, the mix of NOL bacteria (NOL01, NOL02, NOL03 and NOL11) is mixed manually for 5 minutes in the food composition to be tested so as to achieve a minimum concentration of 1×10⁶ CFU/g in Bacillus subtilis (NOL01-NOL03) and minimum 1×10⁶ CFU/g in Lactococcus lactis (NOL11). For each sample, 15 g of raw food matrix is weighed (Precision Series, Fisher Scientific), in a sterile 40 mL jar (Sarstedt, Nümbreccht, Germany).

To prepare the control samples, 15 g of raw food composition to be tested is weighed (Precision Series, Fisher Scientific), in a sterile 40 mL jar (Sarstedt, Nümbreccht, Germany).

### A. Surface Preparation

Flat, sterile petri dishes made of smooth polystyrene, 14 cm in diameter (Sarstedt, Nümbreccht, Germany), considered representative of the surfaces encountered in cattle feed factories, are used to prepare the test surfaces.

The dishes are manually swabbed (Sodibox, Névez, France) with 1 mL of solution containing S. enterica or E. coli at 2×10⁴ CFU/mL, to reach a target concentration of approximately 1.3×10⁶ CFU/m² of surface, thus representing a surface tested for contamination by pathogens. Between each passage of the swab, the dish is left to dry under the Microbiological Safety Station (MSS), then swabbed again by turning it a quarter turn clockwise.

The operation is repeated until the 1 mL of solution is completely spread over the dish.

In the context of tests E and F without food composition, the bacterial composition according to the invention was dissolved in 1 mL and applied according to the same experimental protocol, namely swabbing until the 1 mL of solution corresponding to the dose was exhausted of the composition of bacteria according to the invention present in the 15 g of food matrix (tests A, B, C, D)

### A. Application of Food Matrices and Measurements

Each contaminated surface is subject to an application of 15 g of food matrix (control or test) distributed over the dish by horizontal stirring in order to obtain a homogeneous layer over the entire surface. The dishes will then be sprayed manually (Style 1.5, Matabi) with 8 mL of physiological water, thus modeling the incorporation of water during the manufacturing method. Then, the dishes are placed in a drying oven at 25° C. for a period of 4 hours in order to model the action of the food composition containing at least one Bacillus at a retention area recognized by the person skilled in the art as being conducive to pathogen development.

To represent the effect of a food composition containing at least one Bacillus on the control of surface contamination using a sandblaster in an industrial environment, the petri dish closed by a lid and a paraffin seal (Parafilm, Sigma-Aldricht) is then tilted vertically until the complete elimination of the food composition at the sampling zone on the surface, i.e. the upper 75% of the total surface of the petri dish in the vertical position), i.e. up to 4.2 cm in height. FIG. 1 shows the device used.

For each control group and treatment with the composition according to the invention, produced in pairs, 8 dishes are produced in order to be able to measure the evolution of the pathogens immediately after application (4 dishes) and 24 hours after application (4 dishes).

A surface swab (Sodibox, Névez, France) is taken at the sampling surface, i.e.

the upper 75% of the total surface of the petri dish in the vertical position, for each dish according to standard NF EN ISO 18593, before being stored at 4° C. for analysis.

### A. Pathogen Tracking

At each time step, the samples are analyzed in order to count S. enterica or E. coli according to at least one of the following reference methods:
Standard ISO 21528-2:2017 adapted—Microbiology of the food chain—Horizontal method for the detection and enumeration of Enterobacteriaceae—Part 2: Colony-count technique,
Standard ISO 6579-1:2017 Microbiology of the food chain—Horizontal method for the detection, enumeration and serotyping of Salmonella—Part 1: Detection of Salmonella spp., and
Standard ISO 16649-2:2001 Microbiology of human and animal feeding stuffs—Horizontal method for enumeration of beta-glucuronidase-positive Escherichia coli—Part 2: Colony-count technique at 44° C. using 5-bromo-4-chloro-3-indolyl beta-D-glucuronide.

### A. Summary of Results

All of the test results are presented in the table below and [FIG. 6].

TABLE 6

| | | | Mean change difference |
|---|---|---|---|
| | | Final time | Control − Test |
| Matrix | Pathogen | (hours) | (Log 10 CFU/g) |
| Crushed corn | Salmonella | 24 | 1.2 |
| | Typhimurium | | |
| Sow feed | Salmonella | 24 | 3.0 |
| (see Example 1 for | Typhimurium | | |
| composition) | | | |
| No food matrix | Salmonella | 24 | 0 |
| | Typhimurium | | |

In view of the small number of samples during each test, the effect of the treatment with the food according to the invention on the difference in evolution of the quantity of pathogen after 24 h is measured by the Wilcoxon test with a significance threshold set at 5% (p value less than or equal to 0.05), and a trend threshold set at 10% (p-value less than or equal to 0.1). The treatment effect is significant for the 4 tests comprising a food matrix (p-value=0.02857)

From these experiments, the following conclusions can be reached:
regardless of the food matrix used, whether simple (crushed corn) or complex (pig/sow feed), it is found that the composition according to the invention has a significant effect on reducing pathogenic bacteria present on a surface. It even seems notable that the more complex the feed, the greater the effectiveness;
simply spraying the bacteria according to the invention, without food matrix, has no effect. This is due in particular to the fact that without a nutrient supply, the bacteria are not able to multiply in order to exert their inhibiting effect on the growth of pathogenic bacteria.

1-10. (canceled)

11. A method for decontaminating or preventing contamination of surfaces of a device for receiving food, said method comprising a step of bringing a food composition into contact with the surfaces of said device, said food composition comprising at least one strain of bacteria of the genus Bacillus, said strain being dispersed in said food composition.

12. The method according to claim 11, said food composition further comprising one strain of lactic acid bacteria.

13. The method according to claim 11, where said strain of bacteria of the genus Bacillus is present at a rate of at least 10⁴ cfu per gram of the food composition.

14. The method according to claim 11, wherein said strain of bacteria of the genus Bacillus is one of the following strains:
strain NOL01, deposited at the CNCM on Mar. 14, 2012 under number CNCM 1-4606,
strain NOL02, filed at the CNCM on Jan. 21, 2016 under number CNCM 1-5043, and
strain NOL03, deposited at the CNCM on Mar. 14, 2012 under number CNCM 1-4607,
or any of the strains of bacteria of the genus Bacillus belonging to the same operational taxonomic unit, or OTU, as said NOL01 strain, or said NOL02 strain or said NOL03 strain,
or a mixture of two or more of these strains.

15. The method according to claim 12, wherein said strain of lactic acid bacteria is one of the following strains: the NOL11 strain, deposited at the CNCM on Mar. 14, 2012 under number CNCM 1-4609, one of the strains of lactic acid bacteria belonging to the same operational taxonomic unit as said NOL11 strain, and a mixture of two or more of these strains.

16. The method according to claim 11, wherein said composition comprises at least one of the following combinations of bacteria:
NOL01 and NOL11,
NOL02 and NOL11,
NOL03 and NOL11,
NOL01, NOL02 and NOL11,
NOL01, NOL03 and NOL11,
NOL02, NOL03 and NOL11, and
NOL01, NOL02, NOL03 and NOL11.

17. The method according to claim 11, wherein said surfaces are contaminated or contaminable by enterobacteriaceae.

18. A food composition, in which is dispersed at least one strain of bacteria of the genus Bacillus, notably Bacillus subtilis, and optionally comprising at least one strain of lactic acid bacteria.

19. The method according to claim 11, wherein the bacteria of genus Bacillus, is of the species Bacillus subtilis.

20. The method according to claim 17, wherein the Enterobacteriaceae are bacteria of the genus Salmonella, or Escherichia.

21. The method according to claim 17, wherein the Enterobacteriaceae are Salmonella enterica or Escherichia coli.
